(19) United States
(12) Patent Application Publication
(10) Pub. No.: US 2012/0272959 A1
(43) Pub. Date: Nov. 1, 2012

(54) Device for Producing Dry Aerosol (Varians)

(75) Inventors: Nikolai Penskikh, Saint Petersburg (RU); Alina Chervinskaya, Saint Petersburg (RU); Andrei Penskikh, Saint Petersburg (RU)

(73) Assignees: Alina Chervinskaya, Saint Petersburg (RU); Nikolai Penskikh, Saint Petersburg (RU)

(21) Appl. No.: 13/455,278

(22) Filed: Apr. 25, 2012

(30) Foreign Application Priority Data
Apr. 26, 2011 (RU) 2011117191

(51) Int. Cl. A61M 16/10

(52) U.S. Cl. 128/203.15

(57) ABSTRACT

A device for producing dry aerosol, having a casing, an airway with a supply air flow booster, placed in a chamber, connected with an airway through an outlet, a salt mill made in the form of a grinding body with a rotary drive, aerosol concentration detector, connected to a control unit connected to a switch of mill's drive, and in accordance with the invention, an air pipe is fixed in the outbound channel of a chamber, the lower part of which is located atop a grinding body of a mill, and the upper part—in an airway, while on the upper side of an air tube there is an opening directed toward an air flow in an airway, and a gap between a chamber side and mill's grinding body is 3-10 mm, and a gap between a base of a chamber and mills' grinding body is 1-5 mm.

### RELATED APPLICATIONS

This application claims priority to Russian Patent Application No. RU2011117191, filed Apr. 26, 2011, which is incorporated herein by reference in its entirety.

### FIELD OF THE INVENTION

The invention relates to the medical equipment and can be used to create a microclimate with adjustable parameters for treatment and prevention of respiratory diseases in recreation centers, health resorts, as well as in any organization for personnel health improvement and respiratory tract hygiene.

### BACKGROUND OF THE INVENTION

A known device for producing dry aerosol containing a casing, a fan, a tank powder with a mill and an unloading hole, with a tank powder with a mill made in the form of rotating body mounted on a shaft of the fan, and its working part is located in a tank hole and said shaft is equipped with an unbalance (authorship certificate USSR No 1630834 for an invention, cl. A61M13/00, issued 28 Feb. 1991).

The main disadvantage of the known device for producing dry aerosol is the inability to ensure a desired concentration of aerosol in a treatment room.

The closest analogue with respect to the claimed device in term of a technical essence and achieved result is a device for producing dry aerosol containing a casing, an airway with air flow booster; a salt mill made in the form of a grinding body with a rotary drive placed in a chamber connected to an airway, a separator, a compressor, aerosol concentration probe, connected to a control unit connected to a mill driver, where a mill chamber is equipped with at least two supply inlets, spaced along its perimeter, and located on a grinding body and connected through additional airways with a compressor, and a chamber outlet is perpendicular to the longitudinal axis of an airway, where a separator is located on its end (Patent USSR No 1793932 for an invention, cl. A61M13/00, issued 7 Feb. 1993.—a prototype).

The disadvantages of the known device for producing dry aerosol include a complex design, caused by a compressor which is connected through additional airways with two outlets of a mill chamber.

### SUMMARY OF THE INVENTION

The technical effect of the invention consists in creating a new device for dry aerosol production, which has a simplified design and high operational reliability as well as a high quality of the generated aerosol.

The technical effect for the first variant of the invention is achieved in a device for producing dry aerosol, which contains a casing, an airway with a supply air flow booster, placed in a chamber, connected with an airway through an outlet, a salt mill made in the form of a grinding body with a rotary drive, aerosol concentration detector, connected to a control unit connected to a switch of mill's drive, and in accordance with the invention, an air pipe is fixed in the outbound channel of a chamber, the lower part of which is located atop a grinding body of a mill, and the upper part—in an airway, while on the upper side of an air tube there is an opening directed toward an air flow in an airway, and a gap between a chamber side and mill's grinding body is 3-10 mm, and a gap between a base of a chamber and mills' grinding body is 1-5 mm.

In the first variant of the device for producing dry aerosol the following is preferable:

a chamber outlet is perpendicular to a longitudinal axis of an airway;

an upper part of a chamber outlet, located in an airway, is designed as a flange, and its edge is angularly cut, and an opening is directed opposite to the air flow in an airway;

a hole in the top of an air tube is directed at an angle of 30-60 alongside the air flow in an airway;

a chamber has a cylindrical shape;

in a chamber the junction of a side walls and a base and the upper side are of rounded shape.

The technical effect for the second variant of the invention is achieved in a device for producing dry aerosol, which contains a casing, an airway, a supply air flow booster, a salt mill chamber made in the form of a grinding body with a rotary drive, aerosol concentration detector, connected to a control unit connected to a switch of mill drive, and in accordance with the invention, an air booster is installed on the bottom of a double-sided shaft of a rotary drive on the opposite side of a grinding body, and is located in a housing that is attached to the underside of an airway, while atop a chamber and an airway a top hat is installed with a gap in the side and in the top, and in upper side of an airway and at the top of the side of a top hat air holes are made, and a gap between the side of a chamber side wall and a grinding body is 3-10 mm and a gap between the base of a chamber and a grinding body is 1-5 mm.

In the second variant of the device for producing dry aerosol the following is preferable:

a grinding chamber is placed coaxially with an airway;

an airway is designed as a cylindrical cup;

a chamber has a cylindrical shape;

an upper side of the chamber has a central air hole;

a grinding chamber is made with removable base and top side;

a housing of an air booster is designed as a shallow cylinder with open end facets or in the form of a cylindrical cup with holes in a base.

The technical effect for the third variant of the invention is achieved in a device for producing dry aerosol containing a casing, an airway, an air flow booster, a mill chamber connected to an airway, salt mill as a grinding body, mounted on a shaft of a rotary drive, aerosol concentration detector, connected with a control unit connected to a switch of a drive mill, and according to the invention, a supply air tube is located alongside a longitudinal axis of an airway, where a two-sided rotation shaft goes through a longitudinal hole, and the lower part of a supply air pipe is located above a mill grinding body, and the upper part—under an air booster, mounted on a shaft of rotary drive opposite a grinding body, and placed in a housing, connected to the upper side of an airway, and on the upper side of an airway at least one air channel is made, and on the side of an airway there are air holes, a mill chamber is mounted on the underside of an airway, and a gap between the side wall of a chamber and a grinding body is 3-10 mm and a gap between the base of a chamber and a grinding body is 1-5 mm.

In the third variant of a device for producing dry aerosol the following is preferable:

a separator wall is made in the cavity of an airway;

contains an additional external air mixing drive in a medical treatment room;

a mill chamber is made in the form of a removable cylindrical cup;

in a chamber the junction of a side walls and a base is of rounded shape;

a mill grinding body has a cruciform shape;

a housing of an air flow booster is designed as a shallow cylinder with open end facets or in the form of a cylindrical cup with holes in a base.

Comparative analysis of the claimed invention and a prototype shows the presence of the distinctive features in the claimed variants of a device for producing dry aerosol when compared to the closest analogue, what enables to make a conclusion that the proposed solution meets the “novelty” criteria.

The presence of the distinctive features ensures a positive effect, embodied in the creation of three variants of a device for producing dry aerosol, which has a simplified design and a high operational reliability and provides a high quality of the resulting aerosol.

The use of the claimed invention for creation a microclimate with adjustable parameters for the treatment and prevention of respiratory diseases in recreation centers, health resorts, as well as any organization for the improvement of personnel health and respiratory tract hygiene meets the “industrial applicability” criteria.

### BRIEF DESCRIPTION OF THE DRAWINGS

The invention is presented in the drawings, where

FIG. 1 shows a scheme of the first variant of a device for producing dry aerosol,

FIG. 2—a longitudinal section of a mill chamber in the first variant of a device for producing dry aerosol,

FIG. 3 shows a scheme of the second variant of a device for producing dry aerosol,

FIG. 4—a longitudinal section of a mill chamber of the second variant of a device for producing dry aerosol,

FIG. 5 shows a scheme of the third variant of a device for producing dry aerosol,

FIG. 6—a longitudinal section of the third variant of a mill chamber of a device for producing dry aerosol.

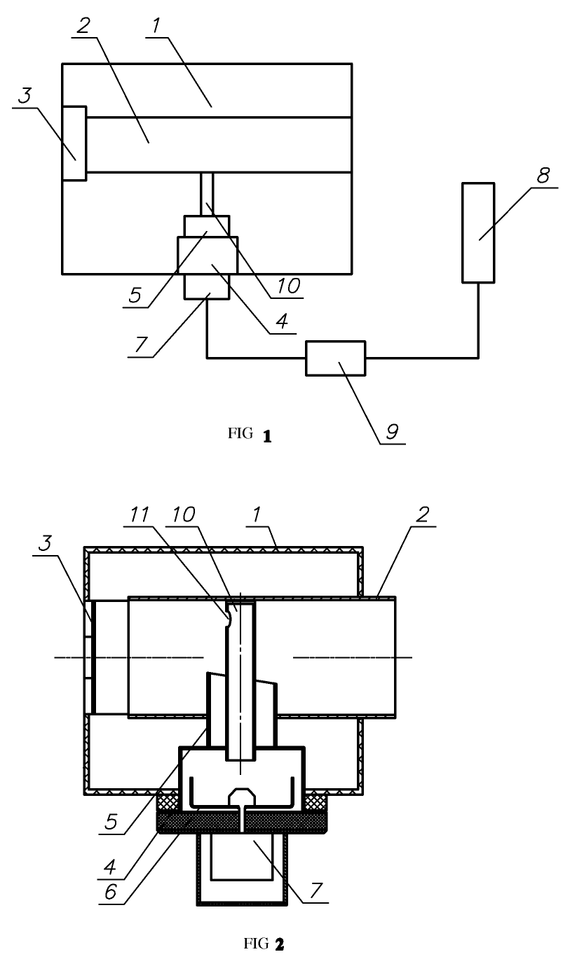

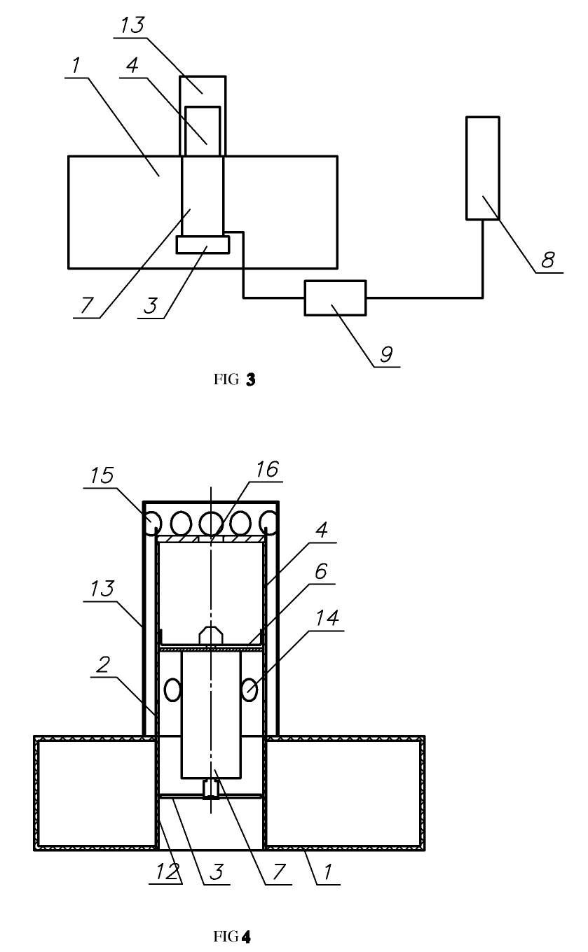

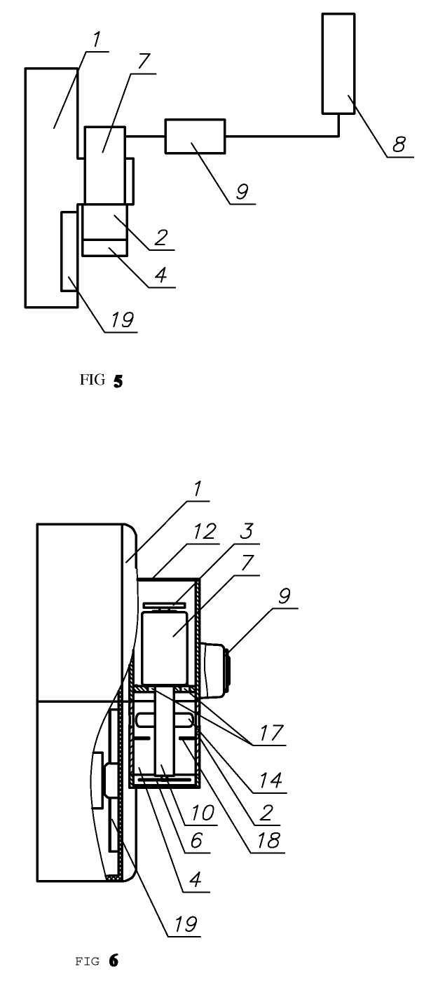

### DETAILED DESCRIPTION OF THE PREFERRED EMBODIMENTS

The first variant of a device for producing dry aerosol comprises a casing 1, an airway 2 with a supply air booster 3, placed in a chamber 4, joined through an outlet 5 with an airway 2, a salt mill as a grinding body 6 with a rotary drive 7, aerosol concentration detector 8, connected to a control unit 9 connected to a switch of mill drive 7, an air tube 10 is fixed in an outbound channel 5 of a chamber 4, its lower part is located atop a grinding body 6 and its upper part—in an airway 2, in the upper side of an air tube 10 there is a hole 11 directed toward an air flow in an airway 2, and a gap between the side wall of a chamber 4 and a grinding body 6 is 3-10 mm and a gap between the underside of a chamber 4 and a grinding body 6 is 1-5 mm.

In the first variant of a device for producing dry aerosol the following is preferable:

an outbound channel 5 in a chamber 4 is perpendicular to a longitudinal axis of an airway 2;

an upper part of an outbound channel 5 in a chamber 4, located in an airway 2, is designed as a flange, angularly cut on its facet, and an opening is opposite to an air flow in an airway 2;

a hole at the top of an air tube 10 is directed at an angle of 30-60° towards an air flow in an airway 2;

a chamber 4 has a cylindrical shape;

in a chamber 4 the junctions of a side walls and a base and the upper side are of rounded shape.

The second variant of the device for producing dry aerosol comprises a casing 1, an airway 2, an air flow booster 3, a chamber 4 with a salt mill as a grinding body 6, with a rotary drive 7, aerosol concentration detector 8, connected to a control unit 9, joined to a switch of a mill drive 7, an air flow booster 3 is installed at the bottom on a two-way shaft of a rotary drive 7 opposite a grinding body 6 and is located in a housing 12 connected to the underside of an airway 2, while above a chamber 4 and an airway 2 a top cap 13 is installed and at the top of a side wall of an airway 2 and the top of a side wall of a top cap 13 air holes 14 and 15 are made, and a gap between a side wall of a chamber 4 and a grinding body 6 is 3-10 mm and a gap between a underside of a chamber 4 and a grinding body 6 is 1-5 mm.

In the second variant of the device for producing dry aerosol the following is preferable:

a grinding chamber 4 is located coaxially with an airway 2;

an airway 2 is made in the form of a cylindrical cup;

a chamber 4 has a cylindrical shape;

an upper side of a chamber 4 has a central air hole 16;

in a chamber 4 the junctions of a side wall and a base is of rounded shape;

a casing 11 of an air flow booster 3 is made in the form of a cylindrical cup.

The third variant of a device for producing dry aerosol comprises a housing 1, an airway 2, an air flow booster, a mill chamber 4, connected to an airway 2, a salt mill as a grinding body 6, installed on a shaft of a rotary drive 7, aerosol concentration detector 8, connected to a control unit 9, connected to a switch of a mill drive 7, along a longitudinal axis of an airway 2 a supply air tube 10 is mounted, through its longitudinal hole goes a two-sided shaft of a rotary drive 7, and the lower part of a supply air tube 10 is located atop a grinding body 6, and its upper part—under an air flow booster 3, mounted on a shaft of a rotary drive 7 opposite a grinding body 6, and located in a housing 12, connected to the upper side of an airway 2, while on the upper side of an airway 2 is at least one air channel 17, and on the side wall of an airway—an air hole 14, a removable mill chamber 4 is mounted on the underside of an airway 2, and a gap between the side wall of a chamber body 4 and a grinding body 6 is 3-10 mm, and a gap between a chamber base 4 and a grinding body 6 is 1-5 mm.

In the second variant of a device for producing dry aerosol the following is preferable:

a separator wall 18 is made in a cavity of an airway 2;

an additional external booster 19 for an air mixing is installed in a medical treatment room;

a mill chamber 4 is designed as a removable cylindrical cup;

in a chamber 4 the junctions of a side wall and a base is of rounded shape;

a grinding body 6 has a cross-shaped grinder;

a housing 12 of an air flow booster 3 is made in the form of a shallow cylinder with an open end facets or in the form of a cylindrical cup with holes in the ground.

The first variant of a device for producing dry aerosol operates as follows:

A salt is poured on the underside of a mill chamber 4. Before the initiation, the parameters on the control unit 9 are set: duration and required aerosol concentration. Under a mill chamber 4 there is a rotary drive 7 in the form of the electric engine, and a specially shaped grinding body 6 is mounted on its shaft. An air is supplied by an air flow booster 3, made in the form of a fan, to an airway 2, where the upper part of a supply air tube 10 is located, that has at its top side an opening 11, directed towards an air flow. Part of an air through an air tube 10 goes into a mill chamber 4 where it is mixed with salt particles. A mix of an air and aerosol due to an air pressure in a supply air tube 10, and as well as due to an ejection force that emerge above an outbound channel 4 of a mill chamber 5 when an air goes through an airway 2, rises up and is being separated due to an impact in the upper part of a mill chamber 4, and out of a mill chamber 4 a fine-dispersed aerosol through an output channel 5 of a mill chamber 4 enters an airway 2, where its coarse (heavy) fraction is separated. After separation of a coarse fraction a fine-dispersed aerosols under the impact of an air flow from an air flow booster 3, made as an electric fan, flows in a medical treatment room. Aerosol concentration detector 8 measures fluctuations of aerosol concentration in a treatment room. A signal from a detector 8 is compared with concentration value set at the beginning of a session. Management of aerosol treatment is carried out by a controller. Depending on the results of a comparison, a mill switches on or off, and an aerosol is supplied or prevented to supply into a treatment room.

The second variant of a device for producing dry aerosol operates as follows:

A salt is poured on the underside of a mill chamber 4, and the parameters on the control unit 9 are set: duration and aerosol concentration control mode. Salt milling and aerosol generation occur during rotation of a grinding body 6, which is located on an axis of a rotary drive 7, made as an electric engine. Poly-dispersed aerosol flows in an airway 2, where its useful (respirable) fraction is separated from a coarse fraction, and under an impact of an air flow from an air flow booster 3, located on an engine shaft on the opposite side to a grinding body 6, through a gap between an airway 2 and a chamber 4 from one side and a top cap 13 on the other side is supplied into a medical treatment room through air hole 15 in a top cap 13. Aerosol concentration management in a treatment room is done as follows. An aerosol concentration detector 9 is installed in a treatment room. Signals from a detector 8 are transferred to a control unit 9, where it is compared with set parameters. Depending on the results of a comparison a signal is generated to turn on or off a mill drive 7.

The third variant of a device for producing dry aerosol operates as follows:

Parameters on a control unit 9 are set: duration and operation mode of a device for aerosol concentration. Salt milling and aerosol generation occur during rotation of a grinding body 6, which is fixed to the bottom of a shaft of a rotary drive 7. Part of an air supplied by an air booster 3 into a mill chamber 4 goes to a grinding zone through an air supply tube 10 and an air channel 17, located on the upper side of an airway 2. Poly-dispersed aerosol flows from a mill chamber 4 into an airway 2, under an impact of an air flow from an air supply tube 10. A useful (respirable) aerosol fraction is separated from its coarse fraction with a special separator wall 18. Fine-dispersed aerosol through air hole 12 is ejected from an airway 2, and further with an air flow from an external booster 19 is supplied into a treatment room.

The stated above advantages superiorly distinguish the claimed invention from a prototype.

What is claimed is:

1. A device for producing dry aerosol comprising a casing, an airway with a supply air flow booster, a salt mill as a grinding body with a rotary drive, placed in a chamber, combined through an outbound channel with an airway, an aerosol concentration probe, connected to a control unit, connected to a switch of a mill drive;
distinguished with a supply air tube mounted in a outbound channel in a chamber, where its lower part is located above a grinding body of a salt mill, and the upper part—in a airway, and a hole in the upper side of an air tube directed towards the air flow in an airway, and a gap between the side wall of a chamber and a grinding body is 3-10 mm and a gap between the base of a chamber and a grinding body is 1-5 mm.

2. The device for producing dry aerosol according to claim 1, wherein an outbound channel of a chamber is perpendicular to the longitudinal axis of an airway.

3. The device for producing dry aerosol according to claim 1, wherein an upper part of an outbound channel of a chamber, located in an airway, is designed as a flange and on its end an angular cut is made, and an opening is directed opposite to an air flow in an airway.

4. The device for producing dry aerosol according to claim 1, wherein a hole in an upper part of an air tube is directed at an angle of 30-60° alongside an air flow in an airway.

5. The device for producing dry aerosol according to claim 1, wherein a chamber has a cylindrical shape.

6. The device for producing dry aerosol according to claim 1, wherein the junctions of a side wall with an upper side and a base in a chamber have rounded shape.

7. A device for producing dry aerosol containing a casing, an airway, an air flow booster, a salt mill, placed in a chamber, as a grinding body with a rotary drive, aerosol concentration detector, connected to a control unit connected to a switch of a mill drive, wherein an air flow booster is installed at the bottom of a double-sided shaft of a rotary drive, opposite to a grinding body, and is located in a housing, connected to the underside of an airway, and a top hat is placed above a chamber and an airway, and air holes are made on the upper part of an airway side wall and on the upper part of a side wall of a top hat, with a gap between the sides wall of a chamber and a grinding body is 3-10 mm and a gap between the base of a chamber and a grinding body is 1-5 mm.

8. The device for producing dry aerosol according to claim 7, wherein a mill chamber is placed coaxially with an airway.

9. The device for producing dry aerosol according to claim 8, wherein an airway is made in the form of a cylindrical cup.

10. The device for producing dry aerosol according to claim 7, wherein a chamber has a cylindrical shape.

11. The device for producing dry aerosol according to claim 7, wherein the upper side of a chamber is made with a central hole in an airway.

12. The device for producing dry aerosol according to claim 7, wherein a chamber is made with removable top side and bottom.

13. The device for producing dry aerosol according to claim 7, wherein a housing of an air flow booster is designed as a shallow cylinder with open end facets or in the form of a cylindrical cup with holes in the ground.

14. A device for producing dry aerosol containing a casing, an airway, an air flow booster, a mill chamber with a salt mill as a grinding body, mounted on a shaft of a rotary drive, aerosol concentration detector, connected to a control unit, connected to a switch of a mill drive, wherein a supply air tube is located alongside a longitudinal axis of an airway, and through its longitudinal opening goes a two-sided shaft of a rotary drive, while the lower part of a supply air tube is located above a mill grinding body, and the upper part—under an air flow booster, mounted on a shaft of a rotary drive opposite a grinding body, and placed in a housing connected to the upper side of an airflow, while on the upper side of an airflow at least one air channel is made, and on the side wall of an airflow—3 air holes are made, a removable mill chamber is mounted on the underside of an airflow, and a gap between the side wall of a chamber and a grinding body is 3-10 mm and a gap between the base of a chamber and a grinding body is 1-5 mm.

15. The device for producing dry aerosol according to claim 14, wherein in a cavity of an airway a wall separator is made.

16. The device for producing dry aerosol according to claim 14, wherein it contains an additional external booster for mixing an air in a treatment room.

17. The device for producing dry aerosol according to claim 14, wherein a mill chamber is made in the form of a removable cylindrical cup.

18. The device for producing dry aerosol according to claim 14, wherein a junction between a side wall of a chamber and a base has a rounded shape.

19. The device for producing dry aerosol according to claim 14, wherein mill grinding body has a cruciform shape.

20. The device for producing dry aerosol according to claim 14, wherein a housing of an air flow booster is designed as a shallow cylinder with an open end facets or in the form of a cylindrical cup with holes in the ground.
